(19) United States
(12) Plant Patent Application Publication
(10) Pub. No.: US 2014/0196182 P1
(43) Pub. Date: Jul. 10, 2014

(54) Blackberry plant named 'Camila'

(50) ;

(71) Applicant: HORTIFRUT S.A., Estero, FL (US)

(72) Inventors: Maria Pilar Banados Ortiz, San Fernando (CL); Alejandra Andrea Salgado Rojas, Santiago (CL); John Reuben Clark, Fayetteville, AR (US)

(73) Assignee: Hortifrut S.A., Estero, FL (US)

(21) Appl. No.: 13/694,787

(22) Filed: Jan. 4, 2013

(51) Int. Cl. A01H 5/00

(52) U.S. Cl. PLT/203

(57) ABSTRACT

A new and distinct primocane-fruiting cultivar of Blackberry plant named “Camila” as described and shown herein. Distinctive characteristics of “Camila” include its earliness, thornless stems, large sized fruit and sweetness without bitter aftertaste.

### BACKGROUND AND SUMMARY

Blackberries are a well-known, aggregate fruit enjoyed by many throughout the world. One example of an existing blackberry variety is “APF-8”, which is marketed as “Prime Jan®”, U.S. Plant Pat. No. 15,788. Other examples of existing, patented blackberry varieties are “Navaho”, U.S. Plant Pat. No. 6,679 and “APF-12”, U.S. Plant Pat. No. 16,989 which is marketed as “Prime Jim®”.

Compared to APF-8 based on some typical results, the present cultivar, Camila (also known as “HFM-5”), and APF-8 are both primocane-fruiting blackberries, but Camila bears fruit on primocanes about one (1) week earlier than APF-8. In addition, plants of Camila have a slightly lower density of spines on the stems and the growth rate is somewhat less than that of APF-8. The fruits of Camila are much sweeter, with higher firmness, have almost no bitter aftertaste and have less acidity than APF-8. The average degrees Brix (° Bx) and percent acidity of the juice of primocane fruits of Camila is 15.1° Bx and 0.7%, respectively, versus 9.8° Bx and 1.3% for primocane fruits of APF-8. The postharvest quality of primocane fruits of Camila is also higher than that of the primocane fruits of APF-8: After seven (7) days storage at 5° C., the primocane fruits of Camila are firmer than those of APF-8 with only 27% of berries of the former showing leaking juice, versus 60% leaking fruits in the case of APF-8.

Compared to Navaho based on some typical results, the present cultivar, “Camila”, bears fruits on both floricanes and primocanes, whereas Navaho bears fruits only on floricanes. Accordingly, the following comparisons to Navaho involve floricane fruit of Camila. The vegetative growth of Camila is considerably more vigorous than that of Navaho. The canes of Camila are erect, thick and thorny, whereas those of Navaho are semi-erect, thin, and thornless. Floricane fruits of Camila are 5.9 g and 2.8 cm long versus 3.0 g and 1.5 cm for Navaho. The fruit of Camila matured four (4) weeks earlier than those of Navaho; the date of first ripening of Camila in Central Chile is week 49 versus week 1 for Navaho.

The present cultivar, Camila, provides one or more advantages compared to these and/or other blackberry varieties such as one or more of an early maturity and better blackberry fruit for at least some purposes.

### BRIEF DESCRIPTION OF THE PHOTOGRAPHS

FIG. 1 is a photograph showing primocane flowers of the Blackberry cultivar “Camila”. This photograph was taken on 22 Mar. 2012 (in Chile).

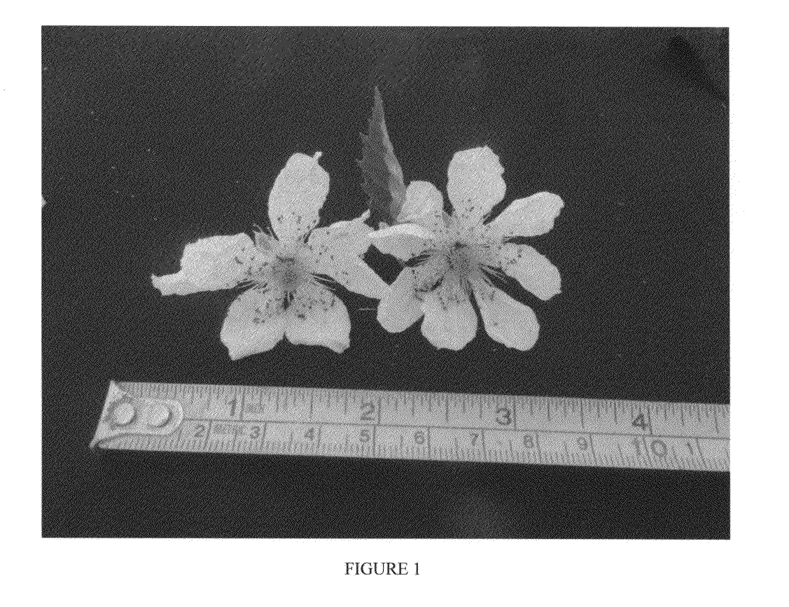

FIG. 2 is a close-up photograph of a primocane shoot of the Blackberry cultivar “Camila” showing primocane color and spine density. This photograph was taken 21 Mar. 2012 (in Chile).

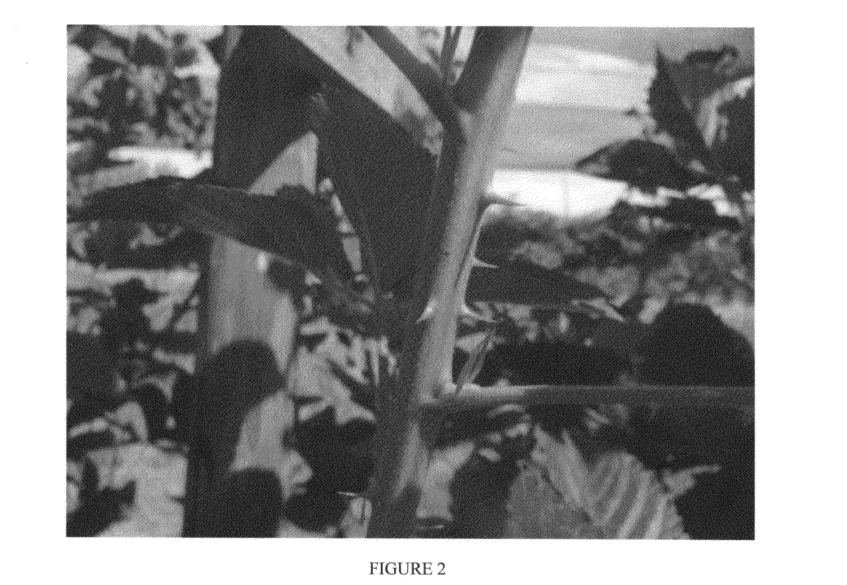

FIG. 3 is a close-up photograph of primocane fruits. This photograph was taken 16 Feb. 2012 (in Chile).

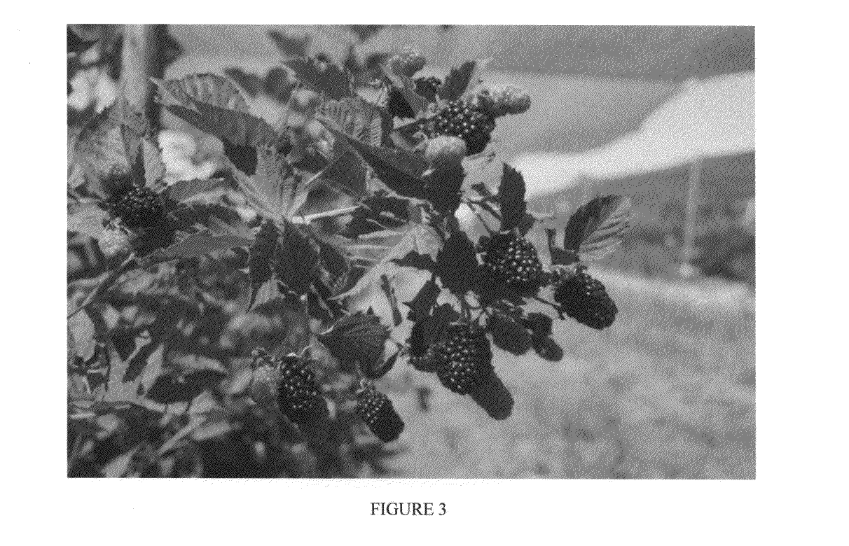

### DETAILED DESCRIPTION

Note: statements of characteristics herein represent exemplary observations of the cultivar herein and will vary depending on time of year, location, annual weather, etc. Where dimensions, sizes, colors, and other characteristics are given, it is to be understood that such characteristics are approximations and averages. The descriptions reported herein are generally from specimen plants that were planted in August 2008 and later at Nogales, Fifth Region, Chile.
Cultivar name: “Camila”
Classification:
   Family.—Rosaceae.
   Botanical name.—Rubus subgenus Rubus.
   Common name.—Blackberry.
Parentage:
Note: The parents and Camila have not been evaluated side-by-side. The data about Camila is from Chile and the data for the parents are from Arkansas, USA.
Female parent:
Name: Name: APF-77 (proprietary, and marketed under the trademark Black Magic™) U.S. Plant patent application (13/374,444) filed 29 Dec. 2011. Comparing APF-77 grown in Arkansas to Camila grown in Chile, the average floricane berry weight of APF-77 is 6 to 7 grams compared to 5.9 grams for Camila. Floricane first bloom date and first ripe date for APF-77 appear to be about a week to 10 days earlier compared to Camila: the floricane first bloom date for APF-77 is 1 April (Northern Hemisphere) versus 10 October (Southern Hemisphere) for Camila while the first ripe date for APF-77 is 2 June versus 8 December for Camila. The first primocane bloom date for APF-77 is 10 June (Northern Hemisphere) compared to 20 December for Camila (Southern Hemisphere), and the first primocane ripe fruit are 15 July (Northern Hemisphere) for APF-77 compared to 10 February (Southern Hemisphere) for Camila. Soluble solids in primocane fruit of Camila is 10.2° Brix compared to 15.1° Brix for APF-77.
Male parent:
Name: APF-109T.
U.S. plant patent: unpatented. Comparing APF-109T grown in Arkansas to Camila grown in Chile, APF-109T is thornless and has a smaller berry than Camila: floricane berries for APF-109T average 4.4 grams versus 5.9 grams for Camila berries. The harvest window appears to be similar or perhaps Camila would be slightly earlier: the window is about 16 June (Northern Hemisphere) for APF-109T compared to about 8 December for Camila (Southern Hemisphere). The soluble solids of Camila are higher: 11.3° Brix for Camila compared to 9.2° Brix for APF-109T. The cross for Camila was made in 2006 near Clarksville, Ark., USA. It was a controlled hand pollination of the female parent (APF 77)×APF 109T (male parent). Resultant seedlings were germinated in a nursery near Hijuelas, Fifth Region of Valparaiso, Chile during the southern hemisphere summer of 2006-07. 285 individual seedlings from this cross were planted in the field near Nogales, Fifth Region of Valpariso, Chile between December 2007 and January 2008. The first evaluation of these seedlings was in early summer (November) 2008 and continued through April 2009. The selection that became Camila was selected in 2009 because the seedling stood out for its very good flavor and the early maturity on primocanes. Camila was first asexually propagated by planting root pieces horizontally in containers containing a bark/peat mix in August 2009 in a greenhouse in Macul, Santiago, Chile. Etiolated shoots that emerged from the root pieces were put in a peat/perlite mix and rooted under humid conditions (covered by clear polyethylene plastic). Camila has also been asexually reproduced using in vitro culture. Propagated plants have retained the original characteristics. Field observations were made in 2009 and later, mostly in Nogales, Chile, including evaluation over three primocane fruiting cycles and two floricane fruiting cycles between January 2009 and April 2011. Camila consistently showed above average horticultural traits and was consistently at the top for taste tests for flavor. For firmness readings, firmness was ranked by on a scale of one to five, with 1 being very firm, and 5 being very soft.
General description: The Camila blackberry is characterized for its early fruit maturity, both on floricanes and on primocanes. The floricane crop begins to ripen during the first week of December (Central Chile). The primocane crop begins to ripen during the second week of February (Central Chile). The fruit has very good (sweet) flavor. The fruit is additionally quite attractive and is elongate and large. Fruit firmness is not very high, but its postharvest keeping ability is acceptable and it shows minimal color change (to red) of the drupelets when put in cold storage. The plant is healthy, moderately vigorous, and productive. Camila is a thorny variety.
Average size information: in the study, plants were “pinched” during growth, so they were not allowed to grow freely. It is estimated that if not pinch-pruned, the primocanes would reach between about 1.7 and 2.2 m in height.
Growth: Plants have medium vegetative vigor, and erect growth habit. Camila makes abundant canes, with primocanes emerging both from the crown of the plant as well as from the roots (as suckers).
Growth rate: The growth rate is moderate, with canes reaching an average of 0.8 meters in height within one month of emergence from the soil in the spring.
Productivity: High, yields average 2.0 kg and 2.9 kg of fruit per meter of row, on the floricane and on the primocane cycles, respectively.
Cold hardiness: Ultimate cold hardiness is unknown, but in Chile dormant plants have resisted midwinter lows of −3° C. without damage.
Branching height of the plants: Unknown (they are always pinched to induce branching and never left to grow to their own devices).
Canes:
General description: Erect, thorny, and medium vigor.
Cane diameter (indicate point of measurement):
Floricane:
   Base.—1.31 cm.
   Midpoint.—1.04 cm.
   Terminal.—0.45 cm.
Immature primocane:
   Base.—0.86 cm.
   Midpoint.—0.67 cm.
   Terminal.—0.33 cm.
Mature primocane:
   Base.—1.25 cm.
   Midpoint.—0.84 cm.
   Terminal.—0.40 cm.
Internode length:
   Base.—11.44 cm.
   Midpoint.—7.68 cm.
   Terminal.—5.01 cm.
Thorn density/30 cm:
   Base.—21.
   Midpoint.—15.
   Terminal.—10.
Primocane color:
   Base.—Light green.
   Midpoint.—Light green.
   Terminal.—Light green.
Floricane color:
   Base.—Green with brownish spots.
   Midpoint.—Green with brownish spots.
   Terminal.—Green with wine-colored spots.
Date of primocane emergence: Primocanes emerge during the first weeks of October (in Chile, at 32° 45′ S. Lat., 220 m elev.) and continue emerging until the second week of November.
Date of budbreak: Vegetative budbreak occurs during the second week of September (in Chile at 32° 45′ S. Lat., 220 m elev.).
Foliage:
General description: Leaves are green with 3 to 5 leaflets, leaves are large with double-serrate margins. The adaxial side of each leaflet is dark green with the veins being somewhat more yellowish. The abaxial side is lighter green than the adaxial side, with soft trichomes (indumentum) over the entire surface. The petioles and petiolules of each leaflet have one rank of thorns, which extend up ¼ of the length of the central vein of each leaflet.
Leaves:
   Width.—16.85 cm.
   Length.—20.13 cm (including petiole).
   Number of leaflets.—3 to 5 per leaf.
Leaflet:
   Width.—5.23 cm.
   Length.—9.46 cm (including petiolules).
   Margin.—Doubly Serrate.
   Shape.—Cordate with acuminate tips.
   Color.—Base Adaxial: Green with the central vein of lighter green color. Base Abaxial: Lighter green than the adaxial side, the central vein being even lighter green with a yellowish cast. Midpoint Adaxial: Green, veins of the same color and shade. Midpoint Abaxial: Green, but of a lighter shade than the adaxial side, yellowish colored veins. Terminal Adaxial: Light green, with the veins being the same color and shade. Terminal Abaxial: Light green, but a lighter shade than the adaxial side, with the veins being the same color.
Petioles:
   Length.—10.25 cm.
   Color.—Light Green (with a yellowish cast).
Petiolules:
   Length.—1-4 cm.
   Color.—Light Green (with a slightly yellowish cast).
Flowers:
Primocane:
   Date of bloom.—(Central Chile). 10% bloom: 20th of December 50% bloom: 30th of December Last bloom: 2nd week of January.
Petal color: Pure White
Reproductive organs:
   Stamens.—The stamens are long, erect, and numerous.
   Pistils.—Numerous.
   Pollen.—Fertile and abundant.
   Ovary.—Superior.
Flower diameter: 3-4 cm.
Petal size:
   Width.—1.2 cm.
   Length.—1.8 cm.
Average number flowers per cluster: 6-7
Average number of petals per flower: 5-9
Peduncle length: 3.58 cm
Peduncle color: Green
Floricane: there are no material differences noted for flower dimensions and inflorescence characteristics for floricanes compared to primocanes, but bloom times for floricanes are:
Date of bloom: (Central Chile)
   10% bloom.—10th of October.
   50% bloom.—20th of October.
   Last bloom.—Last week of October.
Fruit:
General description: The fruit of Camila stands out for its good flavor, large size, and low rate of color regression (to red drupelets) in post-harvest storage. The ratio of soluble solids to acidity averages 19 on floricane fruits and for primocane fruits, the ratio averages 23 (both values on plants grown at the experimental plot at Nogales, Chile). On both the floricane and primocane fruits there is no noticeable bitter aftertaste that is typical of eastern blackberries. The level of reversion of drupelets to a red color in postharvest storage (5° C.) is low. The fruits have an attractive appearance. They are elongated in shape and large in size. The firmness is medium, but it is acceptable.
Primocane:
Average first ripe date: 10th of February (for plants grown under shade cloth at 50% shade) at Nogales, Fifth Region of Valparaiso, Chile. This ripening date is seven (7) days earlier than APF-8 under the same conditions. The primocane harvest lasts for approximately 50 days.
Size: Large (8.4 g on average)
Diameter:
   Equator.—2.05 cm.
   Base pole.—2.11 cm.
   Terminal pole.—1.92 cm.
Length: 3.01 cm
Shape: Oblong (Elongate)
Drupelet size: Medium (0.42 cm average)
Seed size: Small
Firmness: medium
Flavor: Very good, sweet, without bitterness
Soluble solids: 15.1° Bx
pH: Not measured
Acidity: 0.7%
Processed quality: Not evaluated
Uses: Fresh Market
Prickles: None
Floricane:
Average first ripe date: 8th of December (for plants grown under shade cloth at 50% shade) at Nogales, Fifth Region of Valparaiso, Chile. This date is approximately 35 days before Navaho. The floricane harvest lasts for about 35 days.
Size: Medium (5.9 g on average).
Diameter:
   Equator.—2.03 cm.
   Base pole.—21.4 cm.
   Terminal pole.—1.84 cm.
Length: 2.85 cm
Shape: Oblong (Elongate)
Drupelet size: Medium, 0.39 cm
Seed size: Small
Firmness: Medium, similar to the blackberry cultivar APF-12 but firmer than APF-8
Flavor: Sweet and very good flavor
Soluble solids: 11.3° Bx
pH: Not measured
Acidity: 0.6%
Processed quality: Not evaluated
Uses: Fresh Market
Prickles: None

Thus, in some aspects, the Camila blackberry is characterized by having early ripening both on floricanes and primocanes. The fruits themselves are also distinctive in that they have excellent flavor, large size, elongated shape and are visually attractive. Furthermore, the fruits have a low rate of color reversion (to red) in cold storage. The plant has good vigor and high productivity. Fruit characteristics are similar on both primocanes and floricanes, except that primocane fruits are sweeter, have higher yields, and are larger than the floricane crop.

What is claimed is:

1. A new and distinct cultivar of Blackberry plant named “Camila” as described and shown herein.
